(19) United States
(12) Patent Application Publication
(10) Pub. No.: US 2022/0357221 A1
(43) Pub. Date: Nov. 10, 2022

(54) PENIS HARDNESS DETECTOR AND DETECTION METHOD THEREOF

(71) Applicant: YI-TING Lin, Yunlin (TW)

(72) Inventor: YI-TING Lin, Yunlin (TW)

(21) Appl. No.: 17/662,226

(22) Filed: May 5, 2022

(30) Foreign Application Priority Data
May 10, 2021 (TW) 110116824

(51) Int. Cl. G01L 1/22; G01L 5/22; A61B 5/00

(52) U.S. Cl. CPC G01L 1/2231; G01L 5/226; A61B 5/7275; A61B 2562/0261

(57) ABSTRACT

A penis hardness detector and a detection method thereof are provided. The penis hardness detector includes a pair of clamps, a belt body, and a servomotor. Each clamp includes a clamping arm, a force sensor and a pair of gears. The force sensor is disposed inside the clamping arm, and each gear is disposed at one end of the force sensor. Two ends of the belt body are respectively connected to another end of the clamping arm opposite to the gear. The servomotor has an axis passing through the gear. The penis hardness detector can obtain penis hardness results by calculating with formulas.

### CROSS REFERENCE TO RELATED APPLICATIONS

This application claims the priority of Taiwan Patent Application No. 110116824, filed on May 10, 2021, titled “PENIS HARDNESS DETECTOR AND DETECTION METHOD THEREOF”, and the disclosure of which is incorporated herein by reference.

### FIELD OF INVENTION

The present disclosure relates to the technical field of detector, and particularly, to a penis hardness detector. The present disclosure also relates to a detection method of the penis hardness detector.

### BACKGROUND OF INVENTION

Erectile dysfunction is one of the types of male sexual dysfunction, which is characterized by the inability of the penis to erect or maintain erection hardness during sexual activity. Erectile dysfunction may cause depression, anxiety, and low self-esteem. Therefore, men with erectile dysfunction should seek medical help.

In the prior art, a physician utilizes a commercially available penis detection device to detect a patient's night erection. For example, a commercially available RigiScan® detection device is used to detect the penis expansion degree. The principle of the RigiScan® detection device is to put a metal ring on the penis for detection. It does not directly measure the hardness of the penis but measures the expansion of the penis. However, the metal ring may cause discomfort to the user. In addition, the penis of patients with erectile dysfunction often presents a phenomenon of non-hard expansion. Thus, merely measuring the expansion of penis may not fully represent the hardness of the penis. Moreover, the degree of expansion may vary with the size of the penis. Therefore, merely measuring the expansion of the penis may not show the exact results of penis hardness. For another example, a NEVA® penis bioconductivity detection device records the changes in the conductivity of the penis during nocturnal sleep through bioelectrical impedance analysis, thereby inferring the degree of erection of the penis. The two detection devices described above do not directly measure the hardness of the penis, and they can only be measured and compared for a long time when sleeping at night in a resting state, to obtain better stability. Moreover, since nocturnal sleep and sexual behavior are quite different, the measured penile hardness during the nocturnal sleep is not fully representative of penile hardness during the sexual behavior.

The Shore hardness tester is originally a widely used standard for material hardness measurement in the industry. The Shore hardness standard is available, may be reset, and has reproducibility. However, the conventional Shore hardness tester has a sharp detection probe, and it is easy to cause injury to human tissue. Moreover, the size of the conventional Shore hardness tester is large, and it is not suitable for hand-held or worn for continuous detection.

Therefore, it is an urgent problem to be solved in the art to develop a portable penis hardness detector that is not disturbed by the expansion degree of the penis and a detection method thereof.

### SUMMARY OF INVENTION

In order to solve the technical problems in the prior art described above, one object of the present disclosure is to provide a penis hardness detector, and the miniaturized structure of the penis hardness detector is convenient for users to carry and use.

Another object of the present disclosure is to provide a detection method of the penis hardness detector. The hardness of the penis may be accurately measured by the calculation of the formula.

In order to achieve the objects described above, the present disclosure provides a penis hardness detector. The penis hardness detector comprises a first clamp, a second clamp, a belt body, and a servomotor. The first clamp comprises a first clamping arm, a first force sensor, a first convex portion, a first transmission gear, and a second transmission gear. The first clamping arm has a Y-shaped structure and has a first end portion and a second end portion. The first end portion has two ends formed with a V-shaped opening, and the second end portion opposites the first end portion. The first force sensor is disposed inside the first clamping arm and between the first end portion and the second end portion. The first convex portion connects to the first clamping arm, and the first convex portion protrudes from the first clamping arm. The first transmission gear and the second transmission gear are respectively disposed at corresponding one of the two ends of the first end portion.

The second clamp comprises a second clamping arm, a second force sensor, a second convex portion, a third transmission gear, and a fourth transmission gear. The second clamping arm has a Y-shaped structure and has a third end portion and a fourth end portion. The third end portion has two ends formed with a V-shaped opening, and the fourth end portion opposites the third end portion. The second force sensor is disposed inside the second clamping arm and between the third end portion and the fourth end portion. The second convex portion connects to the second clamping arm, and the second convex portion protrudes from the second clamping arm. The third transmission gear and the fourth transmission gear are respectively disposed at corresponding one of the two ends of the third end portion. The third transmission gear meshes with the first transmission gear, and the fourth transmission gear meshes with the second transmission gear.The belt body has two ends, and each end of the belt body respectively connects to the second end portion of the first clamping arm and the fourth end portion of the second clamping arm.

The servomotor has an axis, and the axis passes through the first transmission gear, the second transmission gear, the third transmission gear or the transmission gear.

In one embodiment, the penis hardness detector further comprises a housing. The housing accommodates the servomotor and has two parallel grooves. The first transmission gear and the third transmission gear are located in one of the two parallel grooves, and the second transmission gear and the fourth transmission gear are located in another one of the two parallel grooves.

In one embodiment, the penis hardness detector further comprises two binding portions. One of the binding portions is disposed at the second end portion of the first clamping arm, and another one of the binding portions is disposed at the fourth end portion of the second clamping arm. Each of the two binding portions is detachably connected to corresponding one of the two ends of the belt body, respectively.

In one embodiment, the two binding portions are fixing rings. One of the fixing rings is mounted on the second end portion of the first clamping arm, and another on of the fixing rings is mounted on the fourth end portion of the second clamping arm.

In one embodiment, the penis hardness detector further comprises a waterproof element, and the waterproof element covers the penis hardness detector.

In one embodiment, the waterproof member comprises a silicone membrane.

In one embodiment, the first clamp is formed integrally, and the second clamping arm containing the second force sensor is detachably connected to the third end portion of the second clamping arm.

In one embodiment, the penis hardness detector further comprises a pin, and the pin is disposed between the second clamping arm containing the second force sensor and the third end portion of the second clamping arm.

In one embodiment, the penis hardness detector further comprises an electronic device, and the electronic device controls an operation of the servomotor and transmits a penis hardness result data through a network or bluetooth.

In one embodiment, the first convex portion is a circular convex portion or an elliptical convex portion.

In one embodiment, the first clamping arm of the first clamp and the second clamping arm of the second clamp have a rotation angle between 0 degree and 60 degrees.

In one embodiment, the first force sensor and the second force sensor are a load cell or a varistor.

In one embodiment, a measuring range of the load cell is between 0.1 g and 500 g.

In one embodiment, the belt body is an elastic band.

In one embodiment, a thickness of the first clamp, the second clamp, the belt body or the servomotor is between 0.1 mm and 25 mm.

The present disclosure further provided a detection method of the penis hardness detector. The method comprises the steps of:

providing a penis hardness detector described above;

placing each of standards with different Shore hardness in the penis hardness detector;

abutting a penis against the first force sensor and the second force sensor, and performing detection;

obtaining a slope of a linear regression line by use of formula 1, or obtaining a force required for deformation per unit by use of formula 2; and

corresponding the slope of the linear regression line or the force required for deformation per unit to a Shore hardness value of each standard to obtain a comparison table of the slope of the linear regression line or the force required for deformation per unit and each Shore hardness, and finding a corresponding Shore hardness of the penis on the comparison table;

wherein the formula 1 is F=ß₀S+B₁, and wherein F is a force value; ß₀ is the slope of the linear regression line, representing the force required for deformation per unit; S=R×Sin θ, where S is a vertical distance from a center point of the first transmission gear to a center point of the first convex portion; R is a distance from the center point of the first transmission gear to the center point of the first convex portion; and ß₁ is an intercept of the linear regression line; and

wherein the formula 2 is ß=(Fₙ−F₀)/(Sₙ−S₀), and wherein ß is the force required for deformation per unit; Fₙ is in the n^{th} detection, an average value of high-frequency measurement force when the first clamping arm and the second clamping arm are displaced by a certain distance; F₀ is an average value of an initial detection force of the first clamping arm and the second clamping arm; Sₙ is in the n^{th} detection, an average value of a distance from a vertical line of the center point of the first transmission gear to the center point of the first convex portion and a distance from a vertical line of the center point of the second transmission gear to the center point of the second convex portion; and S₀ is at initial detection, an average value of the distance from the vertical line of the center point of the first transmission gear to the center point of the first convex portion and the distance from the vertical line of the center point of the second transmission gear to the center point of the second convex portion.

In one embodiment, a relationship curve between F and S may also be compared with Shore hardness according to the characteristics of the equation by use other approximate equation curves.

The penis hardness detector of the present disclosure generates a reaction force by pressing the first convex portion and the second convex portion against a surface of the penis, and the generated reaction force is then detected by the first force sensor and the second force sensor to obtain the result of penis hardness. Therefore, the penis hardness detector of the present disclosure directly measures the Shore hardness of the penis through a principle of force and deformation of an object, and is not affected by the degree of the expansion of the penis. In addition, the penis hardness detector of the present disclosure may accurately measure when the penis is actuated by other forces. That is, the hardness of the penis may be measured while the penis is stimulated. Moreover, the first convex portion and the second convex portion of the penis hardness detector of the present disclosure may solve the problem that a sharp detection probe used in the prior art causes injury to human tissue. Furthermore, the penis hardness detector of the present disclosure may be miniaturized to facilitate the user to carry and use. In addition, the detection method of the penis hardness detector of the present disclosure may accurately measure the hardness of the penis by the calculation of the formula.

### BRIEF DESCRIPTION OF DRAWINGS

In order to explain the technical solutions of the present disclosure more clearly, the following will briefly introduce the drawings used in the description of the embodiments or the related art. Obviously, the drawings described below are only some embodiments of the present disclosure. For those skilled in the art, other drawings can be obtained based on these drawings without making creative efforts.

FIG. 1 is a sectional view of a penis hardness detector of one embodiment of the present disclosure.

FIG. 2 is a top view of the penis hardness detector of one embodiment of the present disclosure.

FIG. 3 is a stereoscopic view of the penis hardness detector of one embodiment of the present disclosure.

FIG. 4 is a sectional view of the penis hardness detector of another embodiment of the present disclosure.

### DETAILED DESCRIPTION OF PREFERRED EMBODIMENTS

The following describes the embodiment of the present disclosure through specific examples. Those skilled in the field can understand other advantages and effects of the present disclosure from the content disclosed in the present specification. However, the exemplary embodiments disclosed in the present disclosure are merely for illustrative purposes and should not be construed as a limiting the scope of the present disclosure. In other words, the present disclosure can also be implemented or applied by other different specific embodiments, and various details in the present specification can also be modified and changed based on different viewpoints and applications without departing from the concept of the present disclosure.

Unless otherwise described herein, the singular forms “a” and “the” used in the specification and the appended claims of the present disclosure comprise plural entities. Unless otherwise described herein, the term “or” used in the specification and the appended claims of the present disclosure comprises the meaning of “and/or”.

Example: Detection of a user's penis hardness by use of a penis hardness detector

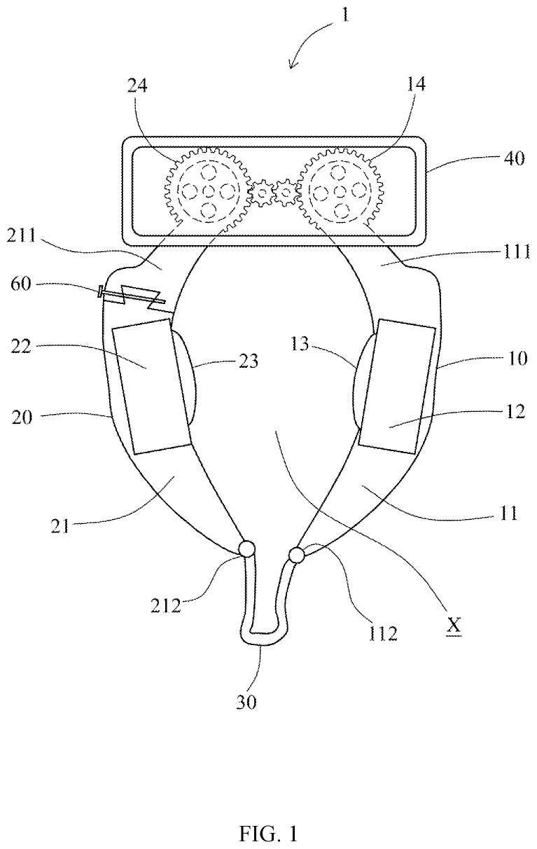

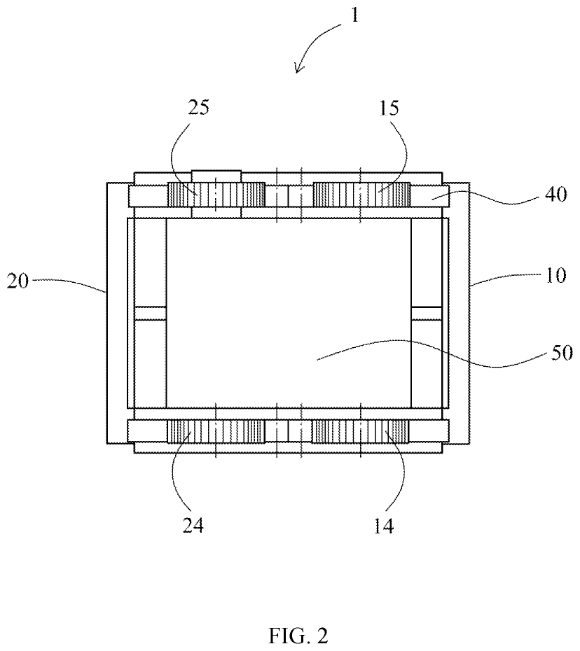

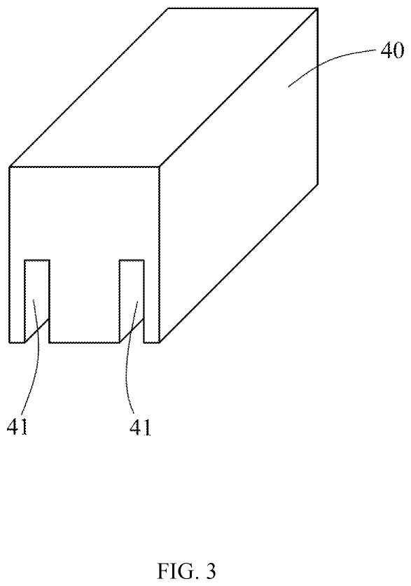

Referring to FIGS. 1 to 4, the penis hardness detector 1 comprises a first clamp 10, a second clamp 20, a belt body 30, a housing 40, a servomotor 50, and a pin 60. The first clamp 10 is integrally formed and has a first clamping arm 11, a first load cell 12, a first convex portion 13, a first transmission gear 14, and a second transmission gear 15. The first clamping arm 11 has a Y-shaped structure and has a first end portion 111 and a second end portion 112. The first end portion 111 has two ends formed with a V-shaped opening, and the second end portion 112 opposites the first end portion 111. The first load cell 12 is disposed inside the first clamping arm 11 and between the first end portion 111 and the second end portion 112. The first convex portion 13 connects to the first clamping arm 11, and the first convex portion 13 protrudes from the first clamping arm 11. The first transmission gear 14 and the second transmission gear 15 are respectively disposed at corresponding one of the two ends of the first end portion 111.

The second clamp 20 has a second clamping arm 21, a second load cell 22, a second convex portion 23, a third transmission gear 24, and a fourth transmission gear 25. The second clamping arm 21 has a Y-shaped structure and has a third end portion 211 and a fourth end portion 212. The third end portion 211 has two ends formed with a V-shaped opening, and the fourth end portion 212 opposites the third end portion 211. The second load cell 22 is disposed inside the second clamping arm 21 and between the third end portion 211 and the fourth end portion 212. The second clamping arm 21 containing the second load cell 22 is detachably connected to the third end portion 211. The second convex portion 23 connects to the second clamping arm 21, and the second convex portion 23 protrudes from the second clamping arm 21 . The third transmission gear 24 and the fourth transmission gear 25 are respectively disposed at corresponding one of the two ends of the third end portion 211. The third transmission gear 24 meshes with the first transmission gear 14, and the fourth transmission gear 25 meshes with the second transmission gear 15.

The belt body 30 is an elastic belt, and each end of the belt body 30 respectively connects to the second end portion 112 of the first clamping arm 11 and the fourth end portion 212 of the second clamping arm 21. In addition, an annular space X is formed around the first clamp 10, the second clamp 20, and the belt body 40.

The housing 40 has two parallel grooves 41, and the housing 40 covers the first transmission gear 14, the second transmission gear 15, the third transmission gear 24, and the fourth transmission gear 25, so that the first transmission gear 14 and the third transmission gear 24 are located in one of the two parallel grooves 41, and the second transmission gear 15 and the fourth transmission gear 25 are located in another one of the two parallel grooves 41.

The servomotor 50 is disposed inside the housing 40 and is electrically connected to the first load cell 12 and the second load cell 22 through a wire. The servomotor 50 has an, and the axis passes through the fourth transmission gear 25. When the servomotor 50 is powered and operated, the fourth transmission gear 25 may be rotated, followed by driving the second transmission gear 15, the first transmission gear 14, and the third transmission gear 24 to rotate in sequence.

The pin 60 is disposed between the second clamping arm 21 containing the second load cell 22 and the third end portion 211, so that the second clamping arm 21 containing the second load cell 22 is detachably connected to the third end portion 211.

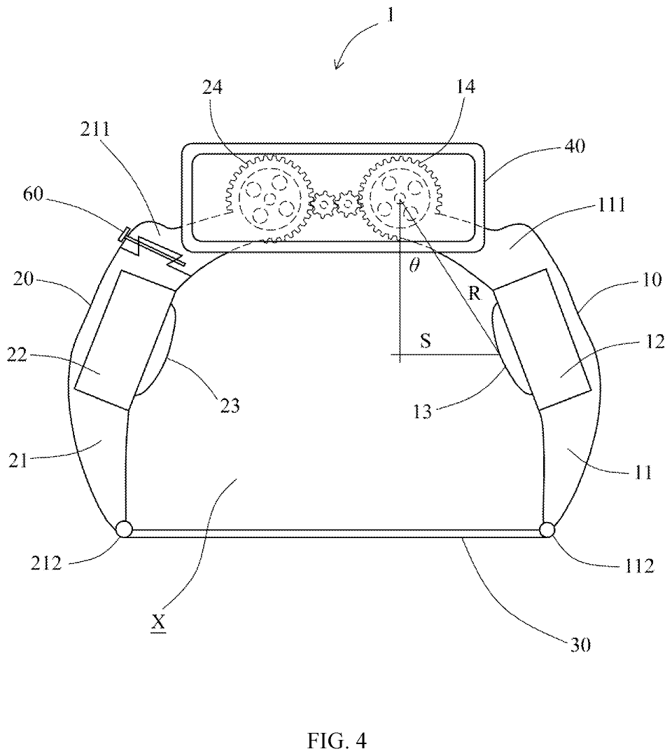

Referring to FIG. 4, before a user uses the penis hardness detector 1 of the present disclosure to detect the penis hardness, each of standards with different Shore hardness is placed in the penis hardness detector 1 to for correction. When the user uses the penis hardness detector 1 of the present disclosure to detect the hardness of the penis, the first clamp 10 and the second clamp 20 move in a direction away from each other, and the penis is placed in the annular space X, allowing the penis to touch first convex portion 13 and the second convex portion 23. The power of the servomotor 50 is then turned on, so that the first clamp 10 and the second clamp 20 are rotated by an angle 0, and the first convex portion 13 and the second convex portion 23 press a surface of the penis to generate a reaction force. The generated reaction force is then detected by the first load cell 12 and the second load cell 22 to obtain a displacement force curve graph. As shown below, a slope of a linear regression line may be obtained by formula 1 of a linear regression method. Alternatively, a force required for deformation per unit may be obtained by formula 2, which divides a difference value between an initial force and a final force by a deformation value of the penis. Finally, the slope of the linear regression line or the force required for deformation per unit may correspond to a Shore hardness value of each standard to obtain a comparison table of the slope of the linear regression line or the force required for deformation per unit and each Shore hardness, and a corresponding Shore hardness of the penis on the comparison table is obtained. The Shore hardness of the penis is then transmitted to the user. This process is automatically completed by the computer program software.

F=ß₀S+ß₁ Formula 1:

In formula 1, F is the force value; ß₀ is the slope of the linear regression line, representing the force required for deformation per unit; S=R×Sin θ, where S is a vertical distance from a center point of the first transmission gear 14 to a center point of the first convex portion 13; R is a distance from the center point of the first transmission gear 14 to the center point of the first convex portion 13; and, ß₁ is an intercept of the linear regression line.

ß=(Fₙ−F₀)/(Sₙ−S₀) Formula 2:

In formula 2, ß is the force required for deformation per unit; Fₙ is in the n^{th} detection, an average value of the high-frequency measurement force when the first clamping arm 11 and the second clamping arm 21 are displaced by a certain distance. The frequency of the high-frequency force measurement is 80 Hz. When the first convex portion 13 and the second convex portion 23 have not touched the penis, the high-frequency force is detected every 1/80 second, and the measured high-frequency force is 0. When the first clamping arm 11 and the second clamping arm 21 start to clamp the penis, which allows the first convex portion 13 and the second convex portion 23 to touch the penis, as the first convex portion 13 and the second convex portion 23 press the surface of the penis, the penis is deformed, thereby generating a reaction force applied to the first load cell 12 and the second load cell 22, and the high-frequency force measurement is continuously detected every 1/80 second and recorded. As the first clamping arm 11 and the second clamping arm 21 move away from each other, the degree of deformation of the penis is reduced, and the reaction force applied to the first load cell 12 and the second load cell 22 is also reduced, and the high-frequency force measurement is continuously detected every 1/80 second and recorded. Finally, the measured high-frequency force values are averaged to obtain an average value of the high-frequency force. F₀ is an average value of an initial detection force of the first clamping arm 11 and the second clamping arm 21. Sₙ is in the n^{th} detection, an average value of a distance from a vertical line of the center point of the first transmission gear 14 to the center point of the first convex portion 13 and a distance from a vertical line of the center point of the second transmission gear 24 to the center point of the second convex portion 23. S₀ is at initial detection, an average value of the distance from the vertical line of the center point of the first transmission gear 14 to the center point of the first convex portion 13 and the distance from the vertical line of the center point of the second transmission gear 24 to the center point of the second convex portion 23.

Moreover, if an emergency occurs when the user uses the penis hardness detector 1 of the present disclosure, the user can immediately remove the pin 60 to disassemble the second clamping arm 21 containing the second load cell 22 and the third end portion 211, and take the penis away from the penis hardness detector 1.

The above provides a detailed introduction to the implementation of the present disclosure, and specific examples are used herein to describe the principles and implementations of the present disclosure, and the description of the implementations above is merely used to help understand the present disclosure. Moreover, for those skilled in the art, according to a concept of the present disclosure, there will be changes in the specific embodiment and the scope of present disclosure. In summary, the content of the specification should not be construed as a limitation to the present disclosure.

What is claimed is:

1. A penis hardness detector, comprising:
a first clamp comprising a first clamping arm, a first force sensor, a first convex portion, a first transmission gear, and a second transmission gear; wherein the first clamping arm has a Y-shaped structure and has a first end portion and a second end portion; the first end portion has two ends formed with a V-shaped opening, and the second end portion opposites the first end portion; the first force sensor is disposed inside the first clamping arm and between the first end portion and the second end portion; the first convex portion connects to the first clamping arm, and the first convex portion protrudes from the first clamping arm; and the first transmission gear and the second transmission gear are respectively disposed at corresponding one of the two ends of the first end portion;
a second clamp comprising a second clamping arm, a second force sensor, a second convex portion, a third transmission gear, and a fourth transmission gear; wherein the second clamping arm has a Y-shaped structure and has a third end portion and a fourth end portion; the third end portion has two ends formed with a V-shaped opening, and the fourth end portion opposites the third end portion; the second force sensor is disposed inside the second clamping arm and between the third end portion and the fourth end portion; the second convex portion connects to the second clamping arm, and the second convex portion protrudes from the second clamping arm; and the third transmission gear and the fourth transmission gear are respectively disposed at corresponding one of the two ends of the third end portion; and wherein the third transmission gear meshes with the first transmission gear, and the fourth transmission gear meshes with the second transmission gear;
a belt body having two ends, wherein each end of the belt body respectively connects to the second end portion of the first clamping arm and the fourth end portion of the second clamping arm; and
a servomotor having an axis, wherein the axis passes through the first transmission gear, the second transmission gear, the third transmission gear or the transmission gear.

2. The penis hardness detector according to claim 1, wherein the penis hardness detector further comprises a housing, the housing accommodates the servomotor and has two parallel grooves, and wherein the first transmission gear and the third transmission gear are located in one of the two parallel grooves, and the second transmission gear and the fourth transmission gear are located in another one of the two parallel grooves.

3. The penis hardness detector according to claim 1, wherein the penis hardness detector further comprises two binding portions; one of the binding portions is disposed at the second end portion of the first clamping arm, and another one of the binding portions is disposed at the fourth end portion of the second clamping arm; and
wherein each of the two binding portions is detachably connected to corresponding one of the two ends of the belt body, respectively.

4. The penis hardness detector according to claim 1, wherein the penis hardness detector further comprises a waterproof element, and the waterproof element covers the penis hardness detector.

5. The penis hardness detector according to claim 1, wherein the waterproof member comprises a silicone membrane.

6. The penis hardness detector according to claim 1, wherein the first clamp is formed integrally, and wherein the second clamping arm containing the second force sensor is detachably connected to the third end portion of the second clamping arm.

7. The penis hardness detector according to claim 1, wherein the penis hardness detector further comprises a pin, and the pin is disposed between the second clamping arm containing the second force sensor and the third end portion of the second clamping arm.

8. The penis hardness detector according to claim 1, wherein the penis hardness detector further comprises an electronic device, and the electronic device controls an operation of the servomotor and transmits a penis hardness result data through a network or bluetooth.

9. The penis hardness detector according to claim 1, wherein the first convex portion is a circular convex portion or an elliptical convex portion.

10. The penis hardness detector according to claim 1, wherein the first clamping arm of the first clamp and the second clamping arm of the second clamp have a rotation angle between 0 degree and 60 degrees.

11. The penis hardness detector according to claim 1, wherein the first force sensor and the second force sensor are a load cell or a varistor.

12. The penis hardness detector according to claim 11, wherein a measuring range of the load cell is between 0.1 g and 500 g.

13. The penis hardness detector according to claim 1, wherein the belt body is an elastic band.

14. The penis hardness detector according to claim 1, wherein a thickness of the first clamp, the second clamp, the belt body or the servomotor is between 0.1 mm and 25 mm.

15. The penis hardness detector according to claim 3, wherein the binding portion is a fixing ring.

16. A detection method of the penis hardness detector, comprising steps of:
providing a penis hardness detector according to claim 1;
placing each of standards with different Shore hardness in the penis hardness detector;
abutting a penis against the first force sensor and the second force sensor, and performing detection;
obtaining a slope of a linear regression line by use of formula 1, or obtaining a force required for deformation per unit by use of formula 2; and
corresponding the slope of the linear regression line or the force required for deformation per unit to a Shore hardness value of each standard to obtain a comparison table of the slope of the linear regression line or the force required for deformation per unit to each Shore hardness, and finding a corresponding Shore hardness of the penis on the comparison table;
wherein the formula 1 is F=ß₀S+ß₁, and wherein F is a force value; ß₀ is the slope of the linear regression line, representing the force required for deformation per unit; S=R×Sin θ, where S is a vertical distance from a center point of the first transmission gear to a center point of the first convex portion; R is a distance from the center point of the first transmission gear to the center point of the first convex portion; and ß₁ is an intercept of the linear regression line; and
wherein the formula 2 is ß=(Fₙ−F₀)/(Sₙ−S₀), and wherein ß is the force required for deformation per unit; Fₙ is in the nth detection, an average value of high-frequency measurement force when the first clamping arm and the second clamping arm are displaced by a certain distance; F₀ is an average value of an initial detection force of the first clamping arm and the second clamping arm; Sₙ is in the n^{th} detection, an average value of a distance from a vertical line of the center point of the first transmission gear to the center point of the first convex portion and a distance from a vertical line of the center point of the second transmission gear to the center point of the second convex portion; and S₀ is at initial detection, an average value of the distance from the vertical line of the center point of the first transmission gear to the center point of the first convex portion and the distance from the vertical line of the center point of the second transmission gear to the center point of the second convex portion.
